(19) United States
(12) Patent Application Publication
(10) Pub. No.: US 2013/0267873 A1
(43) Pub. Date: Oct. 10, 2013

(54) SYSTEMS AND METHODS FOR MONITORING PATIENTS WITH REAL-TIME VIDEO

(71) Applicant: Kenneth J. Fuchs, Wayland, MA (US)

(72) Inventor: Kenneth J. Fuchs, Wayland, MA (US)

(73) Assignee: MINDRAY DS USA, INC., Mahwah, NJ (US)

(21) Appl. No.: 13/443,744

(22) Filed: Apr. 10, 2012

(51) Int. Cl. A61B 5/11; A61B 5/103; H04N 7/18; A61B 5/00

(52) U.S. Cl. 600/595; 600/300; 600/587; 348/77; 348/E07.085

(57) ABSTRACT

A method and system for monitoring remotely located patients that includes real-time video of the remotely located patients. The method and system may be implemented as part of a central nursing station, a remote ICU, or a remote patient monitor. The method and system may also allow for real-time audio communication with the remotely located patients. The real-time video and real-time audio may permit improved response to alarms by allowing medical practitioners to reduce the rate of false-positive and false-negative alarms. Further, the real-time video and real-time audio may be used to generate additional alarm criteria. A camera for generating the real-time video may be part of a patient monitor or may be a separate system. Settings for the camera may be controlled from remotely.

### TECHNICAL FIELD

This disclosure relates generally to systems and methods for monitoring patient data and specifically to systems and methods for monitoring patient data that integrate real-time video with medical parameters.

### BRIEF DESCRIPTION OF THE DRAWINGS

FIG. 1 illustrates a patient monitor equipped with a camera for generating a real-time video signal.

FIG. 2 is an exemplary graphical user interface for a central nursing station that may receive real-time video of a remotely located patient.

FIG. 3 illustrates a computer system for remotely monitoring patients with a selected patient depicted on the display.

FIG. 4 is an exemplary graphical user interface including local patient data, as well as patient data and video from a remotely located patient.

FIG. 5 is a block diagram of one embodiment of a remote patient monitoring system, including a plurality of patient monitors and a central remote monitoring system.

FIG. 6 is a flow chart of a method for displaying patient data and real-time video for a selected patient.

### DETAILED DESCRIPTION

Patient monitors may be used to analyze and display medical parameters obtained from one or more sensors attached to a patient. The medical parameters may include, for example, pulse, temperature, respiration, blood pressure, blood oxygen, electrocardiogram, etc. Medical parameters of a patient may be displayed as a waveform or as a numerical value. A waveform may show the physiological parameter over a period of time, while a numerical value may show the present value of the physiological parameter. Patient monitors may be used by medical practitioners to monitor the condition of patients and to identify alarm conditions based upon monitored medical parameters of a patient. Upon the detection of an alarm condition, an alarm may alert medical practitioners to the condition of a patient requiring immediate attention.

A medical practitioner is often responsible for the care of several patients, many of whom may be in different rooms or wards. It may be difficult or impossible for a single medical practitioner to constantly observe the condition of each patient in person. Instead, the medical practitioner may monitor patients from a central station or individual patient monitors may be equipped to receive and display patient data of remote patients. Additionally, a medical practitioner may sometimes be responsible for the care of distant patients to whom she cannot easily travel or patients scattered among multiple locations. The medical practitioner may remotely monitor the distant patients using medical parameters received from patient monitors for those patients.

Patient monitors, central stations, and remote monitors may be configured to sound an alarm when certain conditions occur. However, simply broadcasting alarms is not a complete solution. For example, the medical practitioner might not be aware that a remote patient monitor has malfunctioned or become disconnected and is no longer transmitting alarms, i.e. a false negative. Alternatively, a malfunction with a sensor or activities by the patient may cause the alarm to sound even though the desired conditions for an alarm are not met, i.e. a false positive.

These problems may be addressed by adding real-time video of the patient to monitoring systems including patient monitors, central nursing stations, and other remote monitors. A medical practitioner may be able to more easily detect a false positive or false negative alarm. In some embodiments, the monitoring systems may allow for audio communication between a patient and a medical practitioner. This allows for further discrimination between false positives, false negatives, and correct alarm states. When a valid alarm is sounded, the audio communication may allow the medical practitioner to appropriately communicate with the alarming patient prior to reaching the patient's bedside. During audio communication, video of the medical practitioner may also be transmitted to the patient. Also, real-time video of the patient may allow for the definition of additional alarm conditions.

The term video, as used herein, is defined broadly. Video includes the detection or measurement of electromagnetic radiation in any region of the electromagnetic spectrum, such as radiation in the infrared, visible, ultraviolet, and x-ray regions. The radiation may be measured by exposing film or sensors in an electromagnetic measurement device to the radiation. Each exposure or measurement may be referred to as an image. The image may comprise pixels representing individual picture elements captured with a particular resolution. Video may or may not require that the images be interlaced. Video does not require that images be captured at any specific rate. Possible rates include, but are not limited to, traditional rates (20-60 Hz) and once per minute or every several minutes (0.001 Hz-0.1 Hz). A video or audio signal is real-time if it is played close in time with the detection of the electromagnetic radiation or sound waves. A video or audio signal may still be close in time and therefore a real-time signal despite delays of several seconds or more.

The embodiments of the disclosure will be best understood by reference to the drawings, wherein like elements are designated by like numerals throughout. In the following description, numerous specific details are provided for a thorough understanding of the embodiments described herein. However, those of skill in the art will recognize that one or more of the specific details may be omitted, or other methods, components, or materials may be used. In some cases, operations are not shown or described in detail in order to avoid obscuring more important aspects of the disclosure.

Furthermore, the described features, operations, or characteristics may be combined in any suitable manner in one or more embodiments. It will also be readily understood that the order of the steps or actions of the methods described in connection with the embodiments disclosed may be changed as would be apparent to those skilled in the art. Thus, any order in the drawings or detailed description is for illustrative purposes only and is not meant to imply a required order, unless specified to require an order.

Embodiments may include various steps, which may be embodied in machine-executable instructions to be executed by a computer system. A computer system comprises one or more general-purpose or special-purpose computers (or other electronic device). Alternatively, the computer system may comprise hardware components that include specific logic for performing the steps or comprise a combination of hardware, software, and/or firmware.

Embodiments may also be provided as a computer program product including a computer-readable medium having stored thereon instructions that may be used to program a computer system or other electronic device to perform the processes described herein. The computer-readable medium may include, but is not limited to: hard drives, floppy diskettes, optical disks, CD-ROMs, DVD-ROMs, ROMs, RAMs, EPROMs, EEPROMs, magnetic or optical cards, solid-state memory devices, or other types of media/computer-readable medium suitable for storing electronic instructions.

Computer systems and the computers in a computer system may be connected via a network. Suitable networks for configuration and/or use as described herein include one or more local area networks, wide area networks, metropolitan area networks, and/or “Internet” or IP networks, such as the World Wide Web, a private Internet, a secure Internet, a value-added network, a virtual private network, an extranet, an intranet, or even standalone machines which communicate with other machines by physical transport of media (a so-called “sneakernet”). In particular, a suitable network may be formed from parts or entireties of two or more other networks, including networks using disparate hardware and network communication technologies.

One suitable network includes a server and several clients; other suitable networks may contain other combinations of servers, clients, and/or peer-to-peer nodes, and a given computer system may function both as a client and as a server. Each network includes at least two computers or computer systems, such as the server and/or clients. A computer system may comprise a workstation, laptop computer, disconnectable mobile computer, server, mainframe, cluster, so-called “network computer” or “thin client,” tablet, smart phone, personal digital assistant or other hand-held computing device, “smart” consumer electronics device or appliance, medical device, or a combination thereof.

The network may include communications or networking software, such as the software available from Novell, Microsoft, Artisoft, and other vendors, and may operate using TCP/IP, SPX, IPX, and other protocols over twisted pair, coaxial, or optical fiber cables, telephone lines, satellites, microwave relays, modulated AC power lines, physical media transfer, and/or other data transmission “wires” known to those of skill in the art. The network may encompass smaller networks and/or be connectable to other networks through a gateway or similar mechanism.

Each computer system includes at least a processor and a memory; computer systems may also include various input devices and/or output devices. The processor may include a general purpose device, such as an Intel®, AMD®, or other “off-the-shelf” microprocessor. The processor may include a special purpose processing device, such as an ASIC, SoC, SiP, FPGA, PAL, PLA, FPLA, PLD, or other customized or programmable device. The memory may include static RAM, dynamic RAM, flash memory, one or more flip-flops, ROM, CD-ROM, disk, tape, magnetic, optical, or other computer storage medium. The input device(s) may include a keyboard, mouse, touch screen, light pen, tablet, microphone, sensor, or other hardware with accompanying firmware and/or software. The output device(s) may include a monitor or other display, printer, speech or text synthesizer, switch, signal line, or other hardware with accompanying firmware and/or software.

The computer systems may be capable of using a floppy drive, tape drive, optical drive, magneto-optical drive, or other means to read a storage medium. A suitable storage medium includes a magnetic, optical, or other computer-readable storage device having a specific physical configuration. Suitable storage devices include floppy disks, hard disks, tape, CD-ROMs, DVDs, PROMs, random access memory, flash memory, and other computer system storage devices. The physical configuration represents data and instructions which cause the computer system to operate in a specific and predefined manner as described herein.

Suitable software to assist in implementing the invention is readily provided by those of skill in the pertinent art(s) using the teachings presented here and programming languages and tools, such as Java, Pascal, C++, C, database languages, APIs, SDKs, assembly, firmware, microcode, and/or other languages and tools. Suitable signal formats may be embodied in analog or digital form, with or without error detection and/or correction bits, packet headers, network addresses in a specific format, and/or other supporting data readily provided by those of skill in the pertinent art(s).

Several aspects of the embodiments described will be illustrated as software modules or components. As used herein, a software module or component may include any type of computer instruction or computer executable code located within a memory device. A software module may, for instance, comprise one or more physical or logical blocks of computer instructions, which may be organized as a routine, program, object, component, data structure, etc., that perform one or more tasks or implement particular abstract data types.

In certain embodiments, a particular software module may comprise disparate instructions stored in different locations of a memory device, different memory devices, or different computers, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices. In addition, data being tied or rendered together in a database record may be resident in the same memory device, or across several memory devices, and may be linked together in fields of a record in a database across a network.

Much of the infrastructure that can be used according to the present invention is already available, such as: general purpose computers; computer programming tools and techniques; computer networks and networking technologies; digital storage media; authentication; access control; and other security tools and techniques provided by public keys, encryption, firewalls, and/or other means.

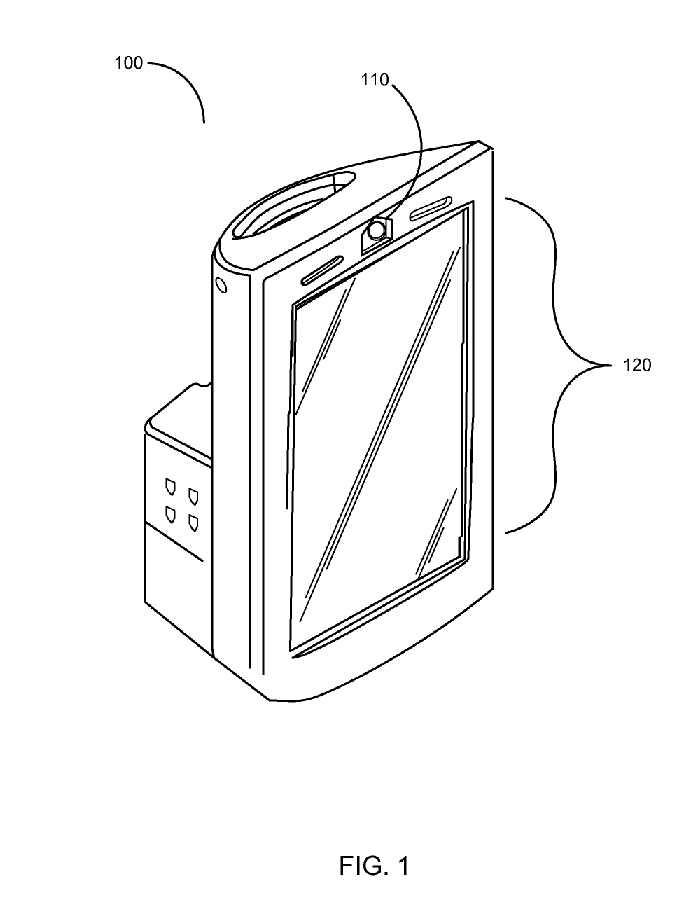

FIG. 1 illustrates a patient monitor 100 comprising a camera 110 for capturing real-time video of a patient. The camera may detect or measure electromagnetic radiation in one or more regions of the electromagnetic spectrum. The patient monitor 100 also receives data signals from sensors connected to the patient. The patient monitor converts those data signals to patient parameter information that can be understood by a medical practitioner, such as patient parameter waveforms, patient parameter numerical values, alarms, and/or other clinically relevant patient data. In other embodiments, some or all of the conversion may occur in the sensors. The patient monitor then displays the patient parameter information on the display 120.

The camera 110 generates a real-time video signal of the patient. In the illustrated embodiment, the camera 110 faces the same direction as the display and is known as a front-facing camera. The camera 110 may also be a rear-facing camera located on the back or side of the patient monitor 100. In some embodiments, the camera 110 may not be incorporated into the body of the patient monitor 100 and may instead connected to the patient monitor 100 in the same manner as other sensors. In other embodiments, the camera 110 may be part of a system entirely separate from the patient monitor. The camera 110 may allow for adjustment of various camera settings. The camera 110 may be able to zoom in and zoom out using an optical or digital zooming feature. The camera 110 may also have the ability to tilt or pan up, down, left, or right, such that it may be directed at an area of interest. The camera 110 may also allow for the focus of the lens to be adjusted, so the real-time video is sufficiently clear to an observer. These adjustments may require manual changes to the camera or may be done automatically through input signals sent to the patient monitor or camera. In other embodiments, the camera may be fixed, and the entire camera system or patient monitor must be moved to change the size of the video and the direction measured by the camera. In some embodiments, the video from the camera 110 may be displayed on the patient monitor 100, such that a medical practitioner may appropriately adjust the camera to display the patient.

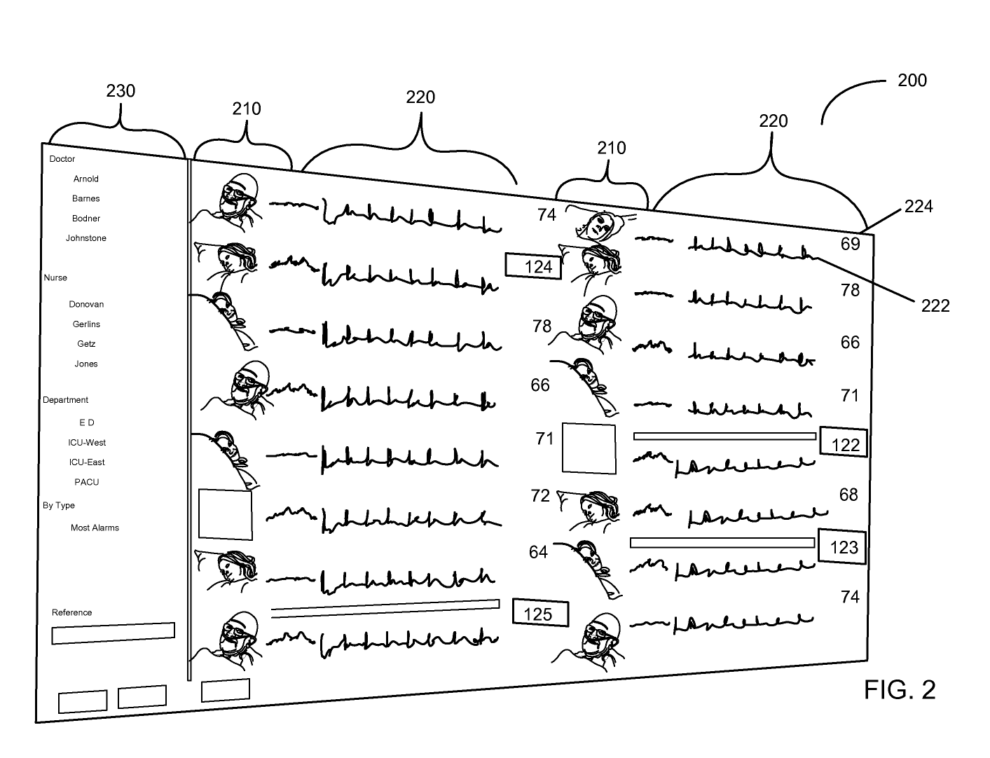

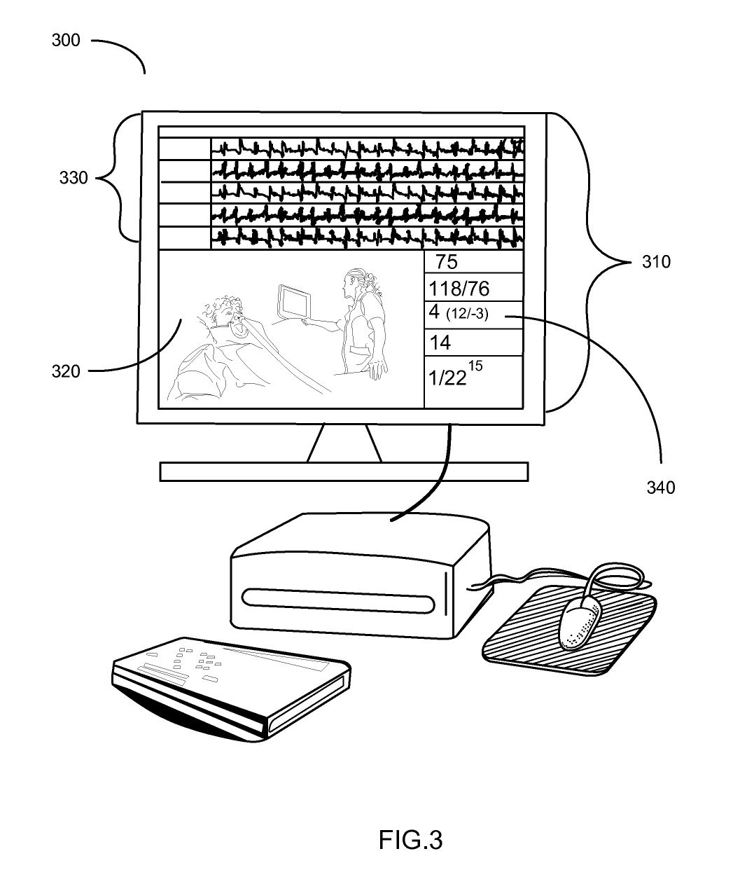

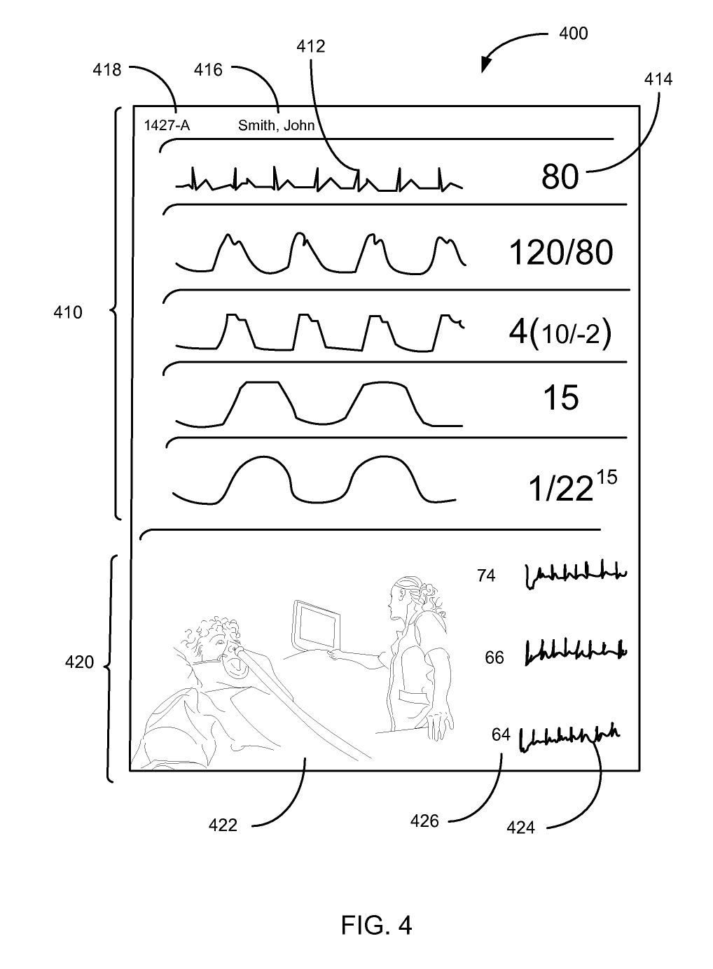

The patient monitor 100 may transmit patient data including the patient parameter information and real-time video signal of the patient to other locations, so the patient may be monitored from a remote monitoring system at a remote location. The patient monitor 100 may also transmit settings that can be remotely adjusted by the remote monitoring system. Examples of remote monitoring systems include a central nursing station for monitoring several patients, as shown in FIG. 2; a remote computer system monitoring a single patient, as illustrated in FIG. 3; and a patient monitor that is viewing local and remote patient data, as depicted in FIG. 4.

The central nursing station 200 of FIG. 2 displays patient data received from remote patient monitors using a graphical user interface. In the illustrated embodiment, only a portion of the patient parameter information 220 for each patient is displayed, such as a cardiographic waveform 222 and a pulse value 224. In other embodiments, all of the patient parameter information may be displayed or a different portion of the patient parameter information may be displayed.

Real-time video 210 of one or more patients may be displayed adjacent to their parameter information 220. The resolution of the real-time video 210 may be reduced to fit the area available in the display. In some embodiments, the real-time video 210 may be located in other positions in the display. All real-time video 210 may be displayed in one area of the display and the parameter information 220 for each patient may be displayed in another area. The location of the real-time video 210 of a patient in the first area may correspond with the location of the parameter information 220 for that patient in the second area. For example, if the real-time video 210 of a patient is in the upper left hand corner of the first area, the parameter information 220 for that patient will be in the upper left hand corner of the second area. In alternative embodiments, the location of the real-time video 210 for a patient is independent of the location of the parameter information 220 for that patient. Some patients may be in a room without a camera. The central nursing station 200 may display static, a black screen, “No Signal,” or any other appropriate image for a patient in a room without a camera.

The central nursing station 200 allows a medical practitioner to choose which patients to display. The medical practitioner may manually choose which patients to display, the station 200 may be configured to always display certain patients (e.g., every patient in a particular ward), or the medical practitioner may be able to choose from various groupings of patients displayed on the monitor in area 230. Groupings may include patients under the care of specific doctors or nurses, patients in specific wards, patients in specific departments, type of patient, or any other grouping known in the art. Patient types may include most alarms.

Patients may also be monitored via the remote computer system 300 of FIG. 3. The remote computer system 300 may be located in an area that is physically remote from the patents. In one embodiment, the remote computer system 300 may be used in a remote ICU where a doctor monitors patients at one or more remotely located hospitals. The remote computer system 300 for monitoring patients may display patient data in a manner similar to that of the central nursing station 200. As discussed above, limited patient parameter information may be displayed in some embodiments. Additionally, the real-time video 210 of individual patients may have a reduced resolution to fit in the space available for the video. A medical practitioner may wish to select a specific patient so that more parameter information and/or a higher resolution video of the patient is displayed. The medical practitioner may wish to do so in response to an alarm.

Once a patient has been selected, the display area 310 may show more detailed patient data for only that patient including an enlarged video 320 of the patient and additional parameter waveforms 330 and/or additional parameter numerical values 340. In the illustrated embodiment, the patient data from other patients is not displayed when a patient is selected. In other embodiments, the additional information 320, 330, 340 is superimposed on the previous display. In still other embodiments, one area of the display shows the additional information 320, 330, 340, while the patient data from other patients is moved to a new location and/or shrunk in size to fit into a second area. All the patient data may be displayed in the second area, or some parameter waveforms or numerical values may be hidden when the patient data is shrunk or moved. Also, parameter numerical values may be displayed instead of parameter waveforms during the shrinking and moving. More than one patient at a time may be selected to display more detailed patient data in some embodiments. The medical practitioner may also be able to remotely control the settings of the camera 110 of the selected patient by using the remote computer system 300 to send signals to the patient monitor 100 or camera 110. The medical practitioner may also be able to alter other settings of the patient monitor 100 that are transmitted to the remote computer system 300.

When a patient is selected, audio communication may be enabled in some embodiments. For full-duplex audio communication, both the patient and medical practitioner will need a microphone or other means of capturing audio signals and a speaker, ear phones, or other means known in the art of playing audio signals. When an alarm sounds, the medical practitioner may then speak with the patient to ask about the patient's condition. Additionally, the medical practitioner may reassure the patient that help is on the way if an emergency situation exists. A medical practitioner may also be able to speak remotely with other medical practitioners present at the scene to provide instructions. In other embodiments, the audio may only be half-duplex or simplex. Additionally, in some embodiments, the initiation of audio communication may be independent of the selection of a patient for more detailed patient data, such as an enlarged video 320. A real-time video signal of the medical practitioner may be transmitted to the patient in some embodiments.

The use of real-time video signals of the patient and real-time audio communication with the patient may allow medical practitioners to better recognize a false positive or false negative alarm. For example, a patient may have dislodged a sensor detecting her pulse, such that an alarm indicates the patient has no pulse. A real-time video signal showing the patient moving may indicate to a medical practitioner that the alarm was in error. Additionally, the patient may respond to audio communication to enable the practitioner to ask questions about the patient's condition. As in other examples, a patient may be in pain, but it is not detected by the attached sensors; or a patient may be choking, but the sensors have yet detected it. A medical practitioner may use the real-time video and/or audio communication to recognize a problem with the patient where an alarm has not been triggered.

In some situations, the medical practitioner may wish to record the parameter information, real-time video, and/or audio. The remote monitoring system may allow a medical practitioner to record the desired patient data. In some embodiments, the remote monitoring system may begin recording parameter information, real-time video, and audio when an alarm begins. In other embodiments, the system may store parameter information, real-time video, and audio for a desired time period after they are received. The system may then save the parameter information, real-time video, and audio before the alarm starts, such that the recording appears to begin before the alarm. A medical practitioner may be allowed to choose to discard an unwanted recording. Alternatively, the medical practitioner must choose to save the recording or it will be discarded after a certain length of time, e.g., 24 hours. In some embodiments, only authorized users have permission to discard or save a recording.

FIG. 4 shows an image that may be displayed on a patient monitor equipped with sensors connected to a patient. The patient parameter information is displayed in a primary display area 410 including patient parameter waveforms 412 and patient parameter numerical values 414. The patient's name 416 and room number 418 may also be displayed. Patient data from one or more remotely located patients may be displayed in secondary display area 420. In this particular embodiment, the real-time video 422 from a selected remote patient is displayed while patient parameter waveforms 424 and patient parameter numerical values 426 from multiple patients are being displayed.

The patient monitor 400 may display the real-time video 422 of one or more remotely located patients in the secondary area 420 during normal operation. Alternatively, the patient monitor 400 may not include the real-time video 422 in the secondary area 420 as part of a normal or default display. Instead, only patient parameter numerical values 426 and/or patient parameter waveforms 424 for remotely located patients may be displayed to protect their privacy. A medical practitioner may need to indicate that she desires to see the real-time video by pressing a button or touching the screen before the real-time video is displayed. The medical practitioner may also need to input an authorization code before being able to view real-time video of remotely located patients.

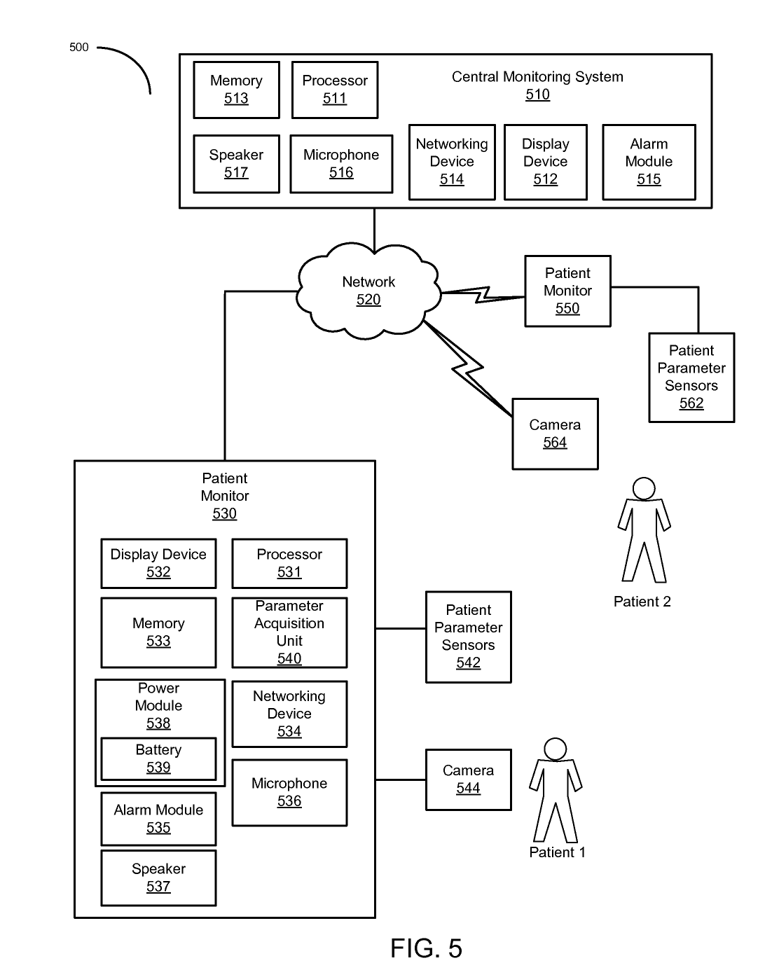

FIG. 5 is a block diagram of a patient monitoring system 500 comprising a central remote monitoring system 510 connected to multiple patient monitors 530, 550 through a computer network 520. One patient monitor 530 is connected to the computer network 520 through a wired connection, while the other patient monitor 550 is connected wirelessly. The patient monitors 530, 550 monitor the patients using patient parameter sensors 542, 562. The patients are also monitored by cameras 544, 564. One camera 544 is connected to the computer network 520 through the patient monitor 530. The other camera 564 is connected directly to the computer network 520 through a wireless signal. The central remote monitoring system 510 may be a central nursing station 200 or a remote computer system 300. The patient monitors 530, 550 may be enabled to display patient data including real-time video of remotely located patients.

The patient monitor 530, according to the illustrated embodiment, includes a processor 531, a display device 532, a memory 533, a networking device 534, an alarm module 535, a parameter acquisition unit 540, a microphone 536, a speaker 537, and a power module 538 connected to a battery 539. The processor 531 is configured to process patient data signals received through the parameter acquisition unit 540 and to display the patient data signals as patient parameter information and/or real-time video on the display device 532. The parameter acquisition unit 540 receives the patient data signals from the patient parameter sensors 542 and camera 544. The parameter acquisition unit 540 may be configured to process the acquired patient data signals in cooperation with the processor 531. The patient monitor 530 may store the patient data signals in the memory 533 along with other data. For example, the patient monitor 530 may store a current set of configuration settings in the memory 533. The power module 538 may be configured to use power from an outlet when the patient monitor 530 is plugged in and to use power from the battery 539 when it is not.

The central remote monitoring system 510 may include a processor 511, a display system 512, a memory 513, a networking unit 514, an alarm module 515, a microphone 516, and a speaker 517. The central remote monitoring system 510 uses the networking unit 514 to receive patient data. The patient data is processed by the processor 511 before being output to a medical practitioner using the display system 512. The networking unit 514 is also used to transmit and receive audio signals from the microphone 516 of the central remote monitoring system 510 to the speaker 537 of the patient monitor 530 and to the speaker 517 of the central remote monitoring system 510 from the microphone 536 of the patient monitor 530.

In one embodiment, the alarm modules 515, 535 detect values of the data signals indicative of problems with the patient. The alarm modules 515, 535 may be situated in only the patient monitor 530, only the central remote monitoring system 510, or both. In some embodiments, the alarm module 535 generates an alarm signal at patient monitor 530, whereas the alarm module 515 may generate an alarm signal at the central remote monitoring system 510 and patient monitors 530, 550. In other embodiments, alarm module 535 may broadcast an alarm to the central remote monitoring system 510 and other patient monitors 550. An alarm may be signaled visually through a change in color or flashing light or audibly through a beep, buzz, siren, voice, or the like. In some embodiments, the alarm may be audible at the central remote monitoring system 510, but only visual at the patient monitors 530, 550.

The alarm modules 515, 535 may be configured for setting alarm conditions that may be detected by the patient monitors 530, 550. In one embodiment, a medical practitioner may specify safe ranges for various medical parameters, outside of which an alarm should be triggered. For instance, the medical practitioner may specify that an alarm should be triggered if a patient's systolic pressure exceeds 180 or drops below 80, or if the patient's diastolic pressure is greater than 100 or less than 50. For certain parameters, the term “range” may be represented as a single value, such as an upper or lower limit. Some alarms may be triggered by a combination of parameters being within particular ranges and/or exceeding or being lower than particular thresholds. The alarm modules 515, 535 may also detect possible medical conditions from the real-time video or real-time audio signals. In some embodiments, only alarm module 515 detects alarm conditions from real-time video.

The alarm modules 515, 535 may analyze the color of the patient's skin in the real-time video in some embodiments. If the patient's lips, extremities, or other body parts turn blue, the alarm modules 515, 535 may signal an alarm. Similarly, if the real-time video captures the infrared region of the electromagnetic spectrum, the alarm modules 515, 535 may monitor the temperature of the patient's body parts via the infrared radiation emitted by those parts. If the temperature of a particular body part drops, the alarm modules 515, 535 may trigger an alarm. Alternatively, the alarm modules 515, 535 may detect if the temperature of a body part has increased or if the patient is flushed. In some embodiments, the alarm modules 515, 535 uses facial or body recognition to distinguish between a patient and background objects. In some embodiments, the alarm modules 515, 535 use the position of the patient's body to determine that the patient is possibly experiencing a medical condition. The alarm modules 515, 535 may detect that a patient has stopped moving, that a patient has fallen, or that a patient is making a gesture indicative of a problem or a request for help.

To detect real-time audio alarms, the alarm modules 515, 535 may use voice recognition to detect the words “help” or “nurse.” The alarm modules 515, 535 may also trigger an alarm if a real-time audio signal is over a certain volume or decibel level. The alarm module 535 may use the microphone 536 to detect alarm conditions even when real-time audio is not being transmitted to central remote monitoring station 510.

Although the embodiment depicted in FIG. 5 includes various distinct software and hardware modules, it is contemplated that, in other embodiments, the functions associated with the various modules may be performed in other ways. Various subsystems may be employed that utilize application specific integrated circuits or other hardware implementations to perform the described functions. Embodiments employing a combination of both hardware and software configured to perform the functionality of the various modules are also contemplated. For example, the functionality of the display system 512, 532 may be performed in part by the processor 511, 531 and/or memory 513, 533. The alarm module 515, 535 may be a software program stored in memory 513, 533 and performed by the processor 511, 531. Further, the functions of various modules illustrated in FIG. 5 may be in other locations or may be distributed throughout the system 500. Alternate embodiments may also include a central server or additional servers that operate as a distributed architecture.

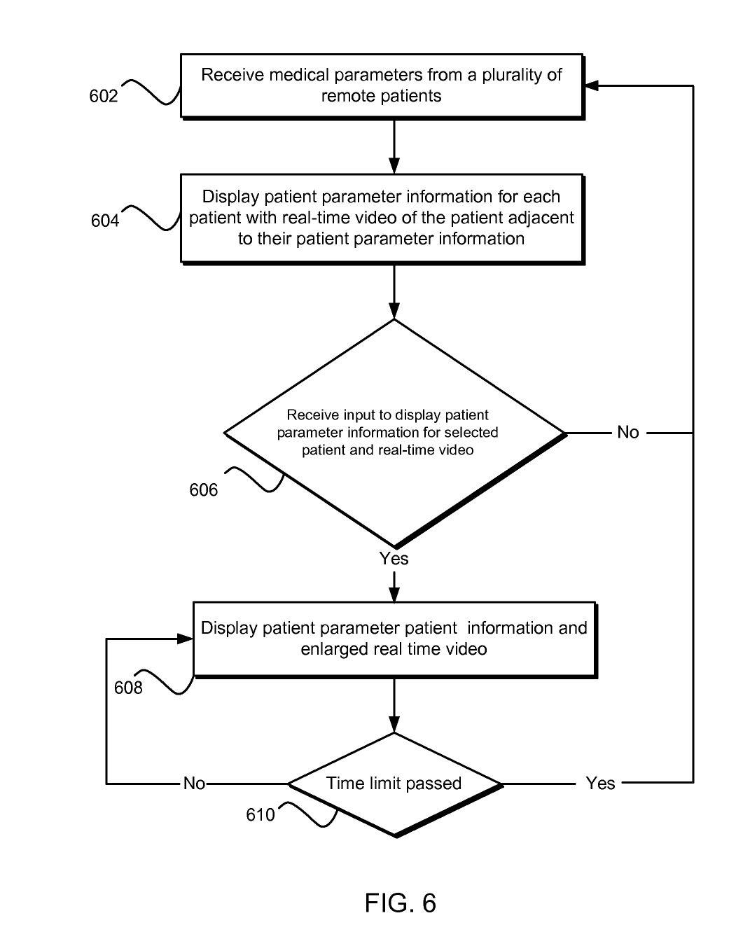

FIG. 6 is a flow chart of a method 600 for displaying medical parameters on a remote monitoring system such as the central remote monitoring system 510 or a remote patient monitor 550. Patient data of one or more remote patients are received 602 from remotely located patient monitors. The patient data may comprise patient parameter information, real-time video, and, in some embodiments, real-time audio. The patient parameter information may include patient parameter waveforms and/or patient parameter numerical values of the patients' pulse, temperature, respiration, blood pressure, blood oxygen level, and electrocardiogram. The remote monitoring system may be designed to receive data from different types of patient monitors 530, 550 (e.g., different models or different manufacturers). The remote monitoring system may appropriately handle patient data in different formats or containing different components.

The remote monitoring system then displays 604 all or a portion of the received remote patient data. The remote monitoring system may display only some patient parameter information; patient parameter information from only some patients, such as those from a grouping in area 230; or no video or a reduced resolution video. In other embodiments, all patient data may be displayed. The remote monitoring system may display different information for different patients based on the format and components of the patient data received.

A medical practitioner may select a particular patient for viewing additional patient data. The medical practitioner may select the patient in response to an alarm. The remote monitoring system may continuously or repeatedly check 606 to see if an input selecting a patient has been received. If so, the remote monitoring system proceeds to displaying 608 additional patient data for the selected patient. Otherwise, the remote monitoring system may return to receiving 602 patient data. The receiving 602, displaying 604, and checking 606 steps may be performed contemporaneously in some embodiments. The remote monitoring system may require authentication information, such as a password or other form of authentication known in the art, before it allows selection of a patient or before it displays additional information for the selected patient.

The remote monitoring system then displays 608 additional information for the selected patient. The additional information may include additional patient parameter information or all patient parameter information. The additional information may also include an enlarged real-time video where the resolution is larger than for real-time video displayed at step 604. An enlarged real-time video may be generated by not reducing the resolution, reducing the resolution less, or interpolating between pixels. The remote monitoring system may also display additional options for the selected patient such as controls to change the camera settings of the remote camera or one or more controls for enabling real-time audio communication and changing audio settings.

A time limit may be specified that limits the amount of time that the additional patient data for the selected remote patient is displayed. The time may begin as soon as the additional information is displayed or when the last input is received from the user. In some embodiments, the start criteria for the time and the time limit may be configurable by the medical practitioner. At 610, the remote monitoring system determines whether the time limit has passed. If the time limit has passed, the remote monitoring system returns to step 602. Otherwise, it may continue to display 608 the additional information. In some embodiments, a medical practitioner may manually indicate that the remote monitoring system should return to step 602. This may be via a “Quit” button or the like.

It will be understood by those having skill in the art that many changes may be made to the details of the above-described embodiments without departing from the underlying principles of the invention. The scope of the present invention should, therefore, be determined only by the following claims.

1. A method for monitoring a plurality of patients, comprising:
receiving, at a computer system, medical parameter data for each patient;
receiving, at the computer system, real-time video signals depicting each patient; and
simultaneously displaying the medical parameter data and the real-time video signals for each patient in a graphical user interface of a display screen.

2. The method of claim 1, wherein the real-time video signals comprise measurements of electromagnetic radiation at visible frequencies.

3. The method of claim 1, wherein the real-time video signals comprise measurements of electromagnetic radiation at infrared frequencies.

4. The method of claim 1, further comprising:
receiving a selection of one of the plurality of patients; and
displaying an enlarged view of the real-time video signals of the selected patient in the graphical user interface.

5. The method of claim 4, further comprising:
displaying additional medical parameter data for the selected patient in the graphical user interface.

6. The method of claim 4, further comprising:
remotely controlling a camera used to capture the real-time video signals of the selected patient in response to a user command.

7. The method of claim 6, wherein remotely controlling comprises at least one of adjusting a zoom level, panning, and tilting the camera.

8. The method of claim 4, wherein displaying the enlarged view comprises covering at least a portion of the medical parameter data and/or real-time video signals for one or more other patients, the method further comprising:
determining that a predetermined amount of time has passed; and
restoring the enlarged view of the real-time video signals of the selected patient to an original size.

9. The method of claim 4, wherein displaying the enlarged view comprises displaying the medical parameter data and real-time video signals for one or more other patients in at least one of a new location and a new size.

10. The method of claim 4, wherein displaying the enlarged view comprises displaying a portion of the medical parameter data and/or real-time video signals for one or more other patients in at least one of a new location and a new size.

11. The method of claim 1, further comprising:
automatically determining from the real-time video signals that a patient is experiencing a possible medical condition; and
generating an audible or visual alert via the computer system to alert a medical professional concerning the possible medical condition.

12. The method of claim 11, wherein automatically determining comprises determining that the patient has changed color.

13. The method of claim 11, wherein automatically determining comprises determining that the patient has stopped moving.

14. The method of claim 11, wherein automatically determining comprises determining that the patient has fallen.

15. The method of claim 11, wherein automatically determining comprises detecting a patient gesture.

16. The method of claim 1, further comprising:
receiving a selection of one of the plurality of patients;
establishing two-way audio communication with the selected patient.

17. A patient monitoring apparatus comprising:
a networking unit configured to receive medical parameter data and real-time video signals for each of a plurality of patients; and
a display system configured to simultaneously display the medical parameter data and the real-time video signals for each patient.

18. The patient monitoring apparatus of claim 17, wherein the real-time video signals for each patient comprise measurements of electromagnetic radiation at visible frequencies.

19. The patient monitoring apparatus of claim 17, wherein the real-time video signals for each patient comprise measurements of electromagnetic radiation at infrared frequencies.

20. The patient monitoring apparatus of claim 17, wherein the display system is further configured to:
receive a selection of one of the plurality of patients; and
display an enlarged view of the real-time video signals of the selected patient.

21. The patient monitoring apparatus of claim 20, wherein the display system is further configured to display additional medical parameter data for the selected patient.

22. The patient monitoring apparatus of claim 20, wherein the networking unit is further configured to remotely control a camera used to capture the real-time video signals of the selected patient in response to a user command.

23. The patient monitoring apparatus of claim 22, wherein the networking unit is further configured to remotely control the camera by at least one of adjusting a zoom level, panning, and tilting the camera.

24. The patient monitoring apparatus of claim 20, wherein the enlarged view covers at least a portion of the medical parameter data and/or real-time video signals for one or more other patients, the display system is further configured to:
determine that a predetermined amount of time has passed; and
restore the enlarged view of the real-time video signals of the selected patient to an original size.

25. The patient monitoring apparatus of claim 20, wherein the display system is further configured to display the enlarged view by displaying the medical parameter data and real-time video signals for one or more other patients in at least one of a new location and a new size.

26. The patient monitoring apparatus of claim 20, wherein the display system is further configured to display the enlarged view by displaying a portion of the medical parameter data and/or real-time video signals for one or more other patients in at least one of a new location and a new size.

27. The patient monitoring apparatus of claim 17, further comprising a processor, wherein the processor is configured to determine automatically from the real-time video signals that a patient is experiencing a possible medical condition and at least one of the display system and an audio system is configured to generate an alert to a medical professional.

28. The patient monitoring apparatus of claim 24, wherein the processor is further configured to determine that the patient has changed color.

29. The patient monitoring apparatus of claim 24, wherein the processor is further configured to determine that the patient has stopped moving.

30. The patient monitoring apparatus of claim 24, wherein the processor is further configured to determine that the patient has fallen.

31. The patient monitoring apparatus of claim 24, wherein the processor is further configured to detect a patient gesture.

32. The patient monitoring apparatus of claim 17, further comprising:
a microphone; and
a speaker, wherein the networking unit is configured to establish two-way audio communication with a selected patient.

33. A patient monitor, comprising:
a parameter acquisition unit configured to acquire local medical parameter data of a local patient;
a networking unit configured to receive medical parameter data and real-time video signals for each of a plurality of patients other than the local patient; and
a display system configured to display the local medical parameter data.

34. The patient monitor of claim 33, wherein the real-time video signals for each patient comprise measurements of electromagnetic radiation in the visible spectrum.

35. The patient monitor of claim 33, wherein the real-time video signals for each patient comprise measurements of electromagnetic radiation in the infrared spectrum.

36. The patient monitor of claim 33, wherein the display system is further configured to:
receive a selection of one of the plurality of patients; and
display the real-time video signals of the selected patient.

37. The patient monitor of claim 36, wherein the display system is further configured to display additional medical parameter data for the selected patient.

38. The patient monitor of claim 36, wherein the networking unit is configured to remotely control a camera used to capture the real-time video signals of the selected patient in response to a user command.

39. The patient monitor of claim 38, wherein the networking unit is further configured to remotely control the camera by at least one of adjusting a zoom level, panning, and tilting the camera.

40. The patient monitor of claim 36, wherein the enlarged view covers at least a portion of the local medical parameter data, the display system is further configured to:
determine that a predetermined amount of time has passed; and
restore display of the local medical parameter data.

41. The patient monitor of claim 36, wherein the display system is further configured to display the enlarged view by displaying the local medical parameter data in at least one of a new location and a new size.

42. The patient monitor of claim 36, wherein the display system is further configured to display the enlarged view by displaying a portion of the local medical parameter data in at least one of a new location and a new size.

43. The patient monitor of claim 33, further comprising a processor, wherein the processor is configured to determine automatically from the real-time video signals that a patient is experiencing a possible medical condition and at least one of the display system and an audio system is configured to generate an alert to a medical professional.

44. The patient monitor of claim 43, wherein the processor is further configured to determine that the patient has changed color.

45. The patient monitor of claim 43, wherein the processor is further configured to determine that the patient has stopped moving.

46. The patient monitor of claim 43, wherein the processor is further configured to determine that the patient has fallen.

47. The patient monitor of claim 43, wherein the processor is further configured to detect a patient gesture.

48. The patient monitor of claim 33, further comprising:
a camera configured to convert electromagnetic radiation measurements to local real-time video signals.

49. The patient monitor of claim 33, further comprising:
a microphone; and
a speaker; wherein the microphone and the speaker are communicatively coupled with the networking unit.

50. A patient monitor, comprising:
a parameter acquisition unit configured to acquire local medical parameter data of a local patient;
an electromagnetic radiation measurement unit configured to convert electromagnetic radiation measurements to local real-time video signals;
a networking unit configured to transmit local medical parameter data and local real-time video signals; and
a display system configured to display the local medical parameter data.
